(19) United States
(12) Patent Application Publication
(10) Pub. No.: US 2023/0151394 A1
(43) Pub. Date: May 18, 2023

(54) BIOLOGIC SYNTHESIS OF DIOLS FROM AMINO ACIDS

(71) Applicant: Taizhou Simplu Technology Co., Ltd., Taizhou City (CN)

(72) Inventors: An-Ping ZENG, Rosengarten (DE); Yongfei LIU, Hamburg (DE)

(73) Assignee: Taizhou Simplu Technology Co., Ltd., Taizhou City (CN)

(21) Appl. No.: 17/916,217

(22) PCT Filed: Mar. 30, 2021
(86) PCT No.: PCT/EP2021/058327

§ 371 Date: Sep. 30, 2022

(30) Foreign Application Priority Data
Mar. 31, 2020 (DE) 10 2020 108 864.7
Mar. 31, 2020 (LU) LU101726

(51) Int. Cl. C12P 7/18; C12N 1/20; C12N 15/52

(52) U.S. Cl. CPC C12P 7/18; C12N 1/20; C12N 15/52; C12R 2001/19

(57) ABSTRACT

A method for the enzymatically catalyzed synthesis of a diol from an amino acid includes enzymatically hydroxylating the amino acid, enzymatically deaminating the amino acid, the deamination resulting in the formation of an oxo group at the position of the amino group, enzymatically decarboxylating the amino acid, and enzymatically hydrogenating the oxo group resulting from the deamination reaction.

### FIELD

The invention relates to the biologic synthesis of diols from amino acids.

### BACKGROUND

Diols, in particular C₂—O₅ diols, are important starting compounds for the production of chemicals like cosmetics and pharmaceuticals (Zhang, Y., Liu, D. & Chen, Z. Production of C2-C4 diols from renewable bioresources: new metabolic pathways and metabolic engineering strategies. Biotechnol Biofuels 10, 299 (2017), doi 10.1186/s13068-017-0992-9). The biotechnological production of diols from renewable resources is therefore of particular interest.

US 2011/014669 A1 describes a biological method for the conversion of L-glutamate to 1,4-butanediol. The method includes (a) conversion of L-glutamate to L-glutamate 5-phosphate, (b) conversion of L-glutamate 5-phosphate to L-glutamate 5-semialdehyde, (c) conversion of L-glutamate 5-semialdehyde to 5-hydroxy-Lnorvaline, (d) conversion of 5-hydroxy-L-norvaline to 5-hydroxy-2-oxopentanoate, (e) conversion of 5-hydroxy-2-oxopentanoate to 4-hydroxybutanal, and (f) the conversion of 4-hydroxybutanal to 1,4-butanediol. This method, however, has disadvantages in that it, for example, involves the production of an instable and toxic intermediate (L-glutamate 5-semialdehyde), is energy-intensive and not generally applicable.

### SUMMARY

It is an object of the invention to provide a reliable and widely applicable method for the production of diols, e.g. from renewable resources.

To solve the above problem the invention provides a method for the biologic synthesis of a diol from an amino acid, the method comprising the steps of

a. Enzymatically hydroxylating the amino acid,

b. Enzymatically deaminating the amino acid, the deamination resulting in the formation of an oxo group at the position of the amino group,

c. Enzymatically decarboxylating the amino acid, and

d. Enzymatically hydrogenating the oxo group resulting from the deamination reaction.

The invention provides a general approach for the production of diols from amino acids and uses enzymatically catalyzed reactions for producing diols from corresponding amino acids.

The term “diol” relates to an organic chemical compound containing two hydroxyl groups (—OH groups).

The term “amino acid” relates to an organic compound containing an amino group (—NH₂) and a carboxyl group (—COOH). The term “alpha amino acid” (α-amino acid) relates to an amino acid having the amino group and the carboxyl group bound to the same C atom (alpha C atom). The amino group can, however, also be attached to another C atom, for example to the beta C atom (“beta amino acid”, β-amino acid), the gamma C atom (“gamma amino acid”, “γ-amino acid”), delta C atom (“delta amino acid”, “δ-amino acid”) or the epsilon C atom (“epsilon amino acid”, “ε-amino acid”), such that the amino group and the carboxyl group are separated by one, two or more C atoms. The term “side chain”, “R group” or “amino acid residue” refers to the part of the amino acid structure that is specific to each amino acid, in case of e.g. an alpha amino acid the carbon chain attached to the alpha C atom. The side chain may be aromatic, aliphatic or acyclic, and may contain hydroxyl or sulfhydryl (thiol) groups. The terms “canonical amino acids” (cAA), “standard amino acids” (SAA) or “proteinogenic amino acids” may be used to denote the 20 amino acids (alpha L amino acids) that are encoded directly by the codons of the genetic code. The twenty canonical amino acids are listed in Table 1 below together with the three-letter code used as abbreviation.

TABLE 1
Canonical amino acids with three-letter code

| | |
|---|---|
| Alanin | Ala |
| Arginin | Arg |
| Asparagin | Asn |
| Asparaginsäure | Asp |
| Cystein | Cys |
| Glutamin | Gln |
| Glutaminsäure | Glu |
| Glycin | Gly |
| Histidin | His |
| Isoleucin | Ile |
| Leucin | Leu |
| Lysin | Lys |
| Methionin | Met |
| Phenylalanin | Phe |
| Prolin | Pro |
| Serin | Ser |
| Threonin | Thr |
| Tryptophan | Trp |
| Tyrosin | Tyr |
| Valin | Val |

The term “non-canonical amino acid” (ncAA), “non-standard amino acid” (nsAA) or “non-proteinogenic amino acid” relates to amino acids other than the above-mentioned canonical amino acids. Examples of non-canonical alpha amino acids are homocysteine (Hcy), Norvaline (Nva), Norleucine (Nle) and Ornithine (Orn).

The expression “Cₙ-Cₘ”, wherein n and m are each positive whole numbers and m is larger than n, signifies a range, which gives the number of C atoms of a compound or residue. The expression expressly includes all intermediate numbers between the limits n and m, respectively independently of each other. The expression “C₁-C₅” (n=1, m=5), thus means a compound, a group or a residue containing 1 to 5, i.e. 1, 2, 3, 4 or 5 C atoms. The expression “C₁-C₅” also includes partial ranges within the range, for example, “C₂-C₄”, i.e. 2, 3 or 4 C atoms, or “C₁-C₄”, i.e. 1, 2, 3 or 4 C atoms, or “C₃-C₅”, i.e. 3, 4 or 5 C atoms. The term “C₂-C₅-diol”, for example, thus refers to a diol having 2, 3, 4 or 5 C atoms.

The term “biologic synthesis of diols from amino acids” relates to the synthesis of diols where all reaction steps converting the amino acid as a starting compound (reactant) to the diol as the product are enzymatically catalyzed. The term encompasses a synthesis using cell-free systems, e.g. isolated enzymes, as well as a synthesis using cellular systems, including the biological or biotechnological synthesis using living organisms, e.g. bacterial cells like Escherichia coli, or yeast cells, or a mixture thereof. Living organisms, for example bacteria, used in the method of the invention, are preferably genetically modified, e.g. to express one or more heterologous enzymes, for example a heterologous hydroxylase or heterologous decarboxylase.

In the context of terms like “enzymatically hydroxylating the amino acid”, “enzymatically deaminating the amino acid”, and “enzymatically decarboxylating the amino acid” the term “amino acid” not only relates to an amino acid as defined above, but to all reaction intermediates in the method of the invention between the amino acid as the reactant and the diol as a product. The term “amino acid” may thus, depending on the order of the reaction steps involved in the method of the invention, for example, also relate to a hydroxylated amino acid, a deaminated amino acid, a decarboxylated amino acid, a deaminated and hydrogenated amino acid, a hydroxylated and decarboxylated amino acid, a hydroxylated and decarboxylated amino acid, a hydroxylated, deaminated and hydrogenated amino acid, a deaminated and decarboxylated amino acid, a deaminated, decarboxylated and hydrogenated amino acid, or to a hydroxylated, decarboxylated and deaminated amino acid.

The term “enzymatically hydroxylating the amino acid” relates to the introduction of a hydroxyl group (—OH group) into an amino acid via a suitable enzyme, e.g. a hydroxylase or dioxygenase, in particular into the amino acid side chain. An example for a suitable hydroxylase is a dioxygenase (MFL or Mfl) from Methylobacillus flagellatus KT (Smirnov, S. V., Sokolov, P. M., Kodera, T., Sugiyama, M., Hibi, M., Shimizu, S., Yokozeki, K. and Ogawa, J. (2012), A novel family of bacterial dioxygenases that catalyse the hydroxylation of free L-amino acids. FEMS Microbiol Lett, 331: 97-104. doi:10.1111/j.1574-6968.2012.02558.x). Other examples are a hydroxylase GriE from Streptomyces DSM 40835 (Zwick I I I, C. R., Renata, H. (2018) Remote C—H Hydroxylation by an alpha-Ketoglutarate-Dependent Dioxygenase Enables Efficient Chemoenzymatic Synthesis of Manzacidin C and Proline Analogs, Journal of the American Chemical Society 140, 1165-1169, doi:10.1021/jacs.7b12918.), GriE from Mycobacterium tuberculosis, or hydroxylases HilA, HilB from Pantoea ananatis AJ13355 (Smirnov S V, Sokolov P M, Kotlyarova V A, Samsonova N N, Kodera T, Sugiyama M, Torii T, Hibi M, Shimizu S, Yokozeki K, Ogawa J. (2013) A novel 1-isoleucine-4′-dioxygenase and 1-isoleucine dihydroxylation cascade in Pantoea ananatis, MicrobiologyOpen 2, 471-481, doi: 10.1002/mbo3.87). The term “hydroxylating the amino acid” not only encompasses the hydroxylation of the amino acid, but also encompasses the hydroxylation of all reaction intermediates between the amino acid as the reactant and the diol as a product. In a case were the hydroxylation step is not the first step of the four steps hydroxylation, deamination, decarboxylation and hydrogenation, acting on an amino acid as defined above, the term also encompasses the hydroxylation of an amino acid that has previously been modified by one or more of the three other steps, i.e. the deamination, decarboxylation and/or hydrogenation step, for example the hydroxylation of an amino acid that has previously been decarboxylated or deaminated, decarboxylated and deaminated or decarboxylated, deaminated and hydrogenated, within the method of the invention.

The term “enzymatically deaminating the amino acid” relates to the removal of an amino group (—NH₂) from an amino acid, in particular to the removal of the amino group attached to the alpha, beta, or gamma C atom of an amino acid, e.g. the amino group attached to the alpha C atom of an alpha L amino acid, catalyzed by a suitable enzyme, e.g. an amino acid deaminase (Aad). Deamination results in an oxo group (═O) at the position of the amino group, i.e. in an oxo group attached to the C atom previously carrying the amino group. The deamination reaction may be oxidative or non-oxidative, preferably oxidative. The term “deaminating the amino acid” encompasses the deamination of the amino acid and of all reaction intermediates between the amino acid as reactant and the diol as a product. The term is thus not to be understood as meaning that the amino acid that is deaminated has to be an unmodified amino acid. In a case were the deamination step is not the first step of the four steps hydroxylation, deamination, decarboxylation and hydrogenation, acting on an amino acid as defined above, the term also encompasses the deamination of an amino acid that has previously been modified by one or more of the three other steps, i.e. the hydroxylation or decarboxylation step, for example the deamination of an amino acid that has previously been hydroxylated or decarboxylated, or has been hydroxylated and decarboxylated, within the method of the invention. A suitable amino acid deaminase is, for example, Aad (also AAD) from Proteus vulgaris (Aadᵥᵤₗ) or Proteus mirabilis (Aadₘᵢᵣ).

The term “enzymatically decarboxylating the amino acid” relates to the removal of the carboxyl group (—COOH) from an amino acid with a suitable decarboxylase (carboxy-lyase). The C atom of the carboxyl group is thus removed from the carbon chain of the amino acid, resulting in a product having one C atom less than the amino acid precursor. An example for a decarboxylase is the alpha keto acid decarboxylase (Kdc). Another example is the α-keto acid decarboxylase PDC from Zymomonas mobilis). The term “decarboxylating the amino acid” encompasses the decarboxylation of the amino acid and of all reaction intermediates between the amino acid as reactant and the diol as a product. The term is thus not to be understood as meaning that the amino acid that is decarboxylated has to be an unmodified amino acid. In a case were the decarboxylation step is not the first step of the four steps hydroxylation, deamination, decarboxylation and hydrogenation, acting on an amino acid as defined above, the term also encompasses the decarboxylation of an amino acid that has previously been modified by one or more of the three other steps, i.e. the hydroxylation, deamination or hydrogenation step, for example the decarboxylation of an amino acid that has previously been hydroxylated or deaminated, or has been hydroxylated, deaminated and hydrogenated, within the method of the invention.

The term “enzymatically hydrogenating the oxo group resulting from the deamination” relates to the formation of a hydroxyl group (—OH) from an oxo group (═O) formed during deamination of the amino group. The terms “reduction of the oxo group” or “reduction of the aldehyde group” may be used synonymously. A suitable enzyme for this step is, for example, the aldehyde reductase (alcohol dehydrogenase) YqhD from E. coli.

The term “heterologous” refers to the foreign origin of an element, for example an enzyme or other protein. “Foreign” means that the element thus does not occur in the target cell, and for example originates from a cell or an organism with different genetic makeup, such as an organism of a different species.

The terms “endogenous” or “homologous” is used herein with respect to an enzyme or protein to refer to it as a native enzyme or protein, i.e. an enzyme or protein naturally occurring in the target cell, in contrast to a heterologous enzyme or protein.

By “expression” is meant here the conversion of a genetic information into a product, for example the formation of a protein or a nucleic acid on the basis of the genetic information. In particular, the term encompasses the biosynthesis of a protein, e.g. an enzyme, based on genetic information including previous processes such as transcription, i.e. the formation of mRNA based on a DNA template. If the term is used here in relation to a protein, e.g. an enzyme, for example in the form “enzyme x is expressed” or “enzyme x is overexpressed”, this means that the associated protein gene is expressed or overexpressed. The term expression encompasses an overexpression, i.e. an expression of a gene that is stronger than the expression of the same gene in the same regulatory context or in its natural context, thus leading to more gene product. Overexpression can be brought about, for example, by introducing additional copies of the gene coding for the gene product into a cell and/or by replacing the natural promoter under whose control the expression of the gene takes place with a stronger promoter. The term “additional copy” can also include that at least one heterologous gene coding for the gene product is introduced.

The amino acid used in the method of the invention can be any amino acid, e.g. an alpha amino acid, a beta amino acid, gamma amino acid or delta amino acid. Preferably, the amino acid is an alpha amino acid, most preferred an alpha L amino acid.

The diol produced is preferably a C₂-C₅ diol, further preferred a C₃-C₅ diol. Examples of such diols are isopentyldiol (IPDO), 1,3-propanediol (1,3-PDO), 1,3-butanediol (1,3-BDO), 1,4-pentanediol (1,4-PTD), 2-methyl-1,3-propanediol (2-M-1,3-PDO, MPO), 2-methyl-1,4-butanediol (2-M-1,4-BDO), 2-methyl-1,3-butanediol (2-M-1,3-BDO), 2-ethyl-1,3-propanediol (2-E-1,3-PDO), 1,3-pentanediol (1,3-PTD), 1,4-butanediol (1,4-BDO).

In the method of the invention the amino acid is preferably enzymatically hydroxylated at a C atom of the side chain. In case of an alpha amino acid it is thus preferred that the hydroxyl group is attached to a C atom other than the alpha C atom. However, in case of, for example, glycine, where there is no carbon side chain but a hydrogen atom attached to the alpha C atom, the hydroxyl group may be attached to the alpha C atom. The hydroxyl group is preferably introduced at the first four C atoms following the alpha C atom, i.e. the beta, gamma, delta or epsilon C atom. For reasons of clarity, it is to be noted that the terms “hydroxylation” or “introduction of a hydroxyl group” do not relate to the deamination and hydrogenation reactions replacing the amino group with a hydroxyl group. The OH group resulting from the latter reactions may also be denoted as the “second hydroxyl group” whereas the hydroxyl group introduced via the enzymatic hydroxylation reaction may be denoted as the “first hydroxyl group”. Further, it is preferred that the hydroxylation takes place at a C atom of the side chain not being part of an aromatic ring.

The method steps a to d may be performed in any order, with the proviso that the deaminating reaction has to precede the hydrogenation (reduction) reaction. It is, however, preferred that the method steps are performed in the order a, b, c, d (hydroxylation-deamination-decarboxylation-hydrogenation). It is preferred that the steps follow each other directly.

In a preferred embodiment of the invention the method thus comprises the steps of
Enzymatically hydroxylating the amino acid,
Enzymatically deaminating the hydroxylated amino acid, forming an oxo group from the amino group of the amino acid,
Enzymatically decarboxylating the hydroxylated and deaminated amino acid, and
Enzymatically hydrogenating the oxo group resulting from the deamination reaction.

In this embodiment the steps carried out correspond to the following order of the above method steps: a, b, c, d. This embodiment is especially preferred since it is thermodynamically favorable, does not involve the production of potential toxic intermediates and does not require expensive co-enzymes.

In a preferred embodiment of the invention the amino acid is an aliphatic (=non-aromatic) amino acid. The method of the invention can, however, also be used to synthesize a diol from an aromatic amino acid, e.g. tryptophan, tyrosine, or phenylalanine. An aromatic amino acid can be hydroxylated at a position outside the aromatic ring or at a position within the aromatic ring.

Examples of diols that can be produced with the method of the invention include the compounds listed in Table 2 below:

TABLE 2
Examples of diols that can be produced with the method of the invention and
corresponding amino acids (canonical and non-canonical) as starting compound.

| No. | Structure | Name | Amino acid as starting compound |
|---|---|---|---|
| 1 | 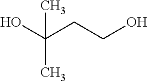 | Isopentyldiol (IPDO) | 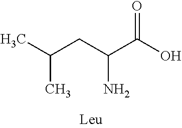 |
| 2 | 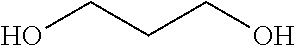 | 1,3-Propanediol (1,3-PDO) | 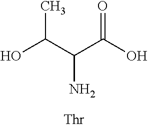 |
| 3 | 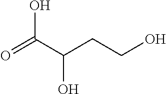 | 2,4-Dihydroxy- butyric acid (2,4-DHB) | 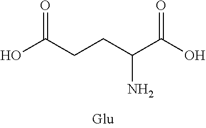 |
| 4 | 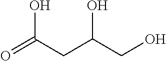 | 3,4-Dihydroxy- butyric acid (3,4-DHBA) | 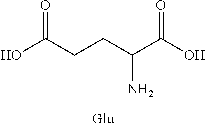 |
| 5 | 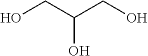 | α-Monothio- glycerol | 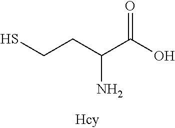 |
| 6 | 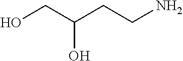 | 4-Amino-1,2- butanediol | 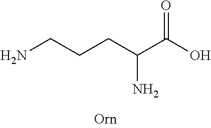 |
| 7 | 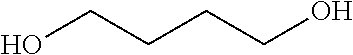 | 1,4-Butanediol (1,4-BDO) | 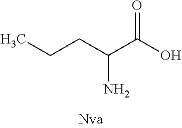 |
| 8 | 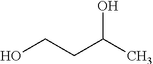 | 1,3-Butanediol (1,3-BDO) | 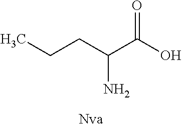 |
| 9 | 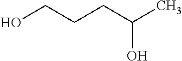 | 1,4-Pentanediol (1,4-PTD) | 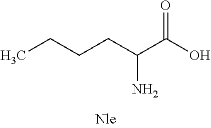 |
| 10 | 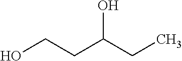 | 1,3-Pentanediol (1,3-PTD) | 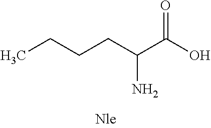 |
| 11 | 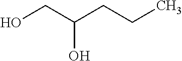 | 1,2-Pentanediol (1,2-PTD) | 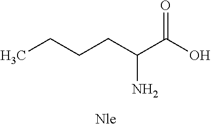 |
| 12 | 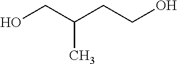 | 2-Methyl-1,4- butanediol (2-M-l,4-BDO) | 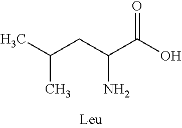 |
| 13 | 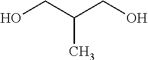 | 2-Methyl-1,3- propanediol (2-M-1,3-PDO; MPO) | 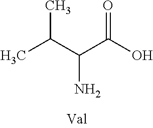 |
| 14 | 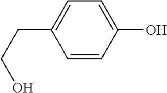 | 4-Hydroxy- phenylethanol | 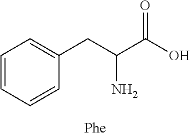 [SMILES: NC(Cc1ccccc1)C(=O)O] |
| 15 | 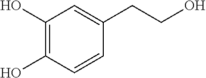 [SMILES: OCCc1ccc(O)c(O)c1] | 3,4-Dihydroxy- phenylethanol | 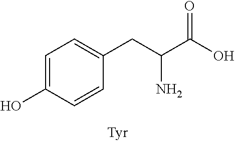 [SMILES: NC(Cc1ccc(O)cc1)C(=O)O] |
| 16 | 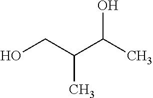 | 2-Methyl-1,3- Butanediol (2-M-1,3-BDO) | 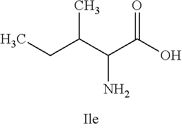 |
| 17 | 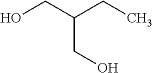 | 2-Ethyl-1,3- propanediol (2-E-1,3-PDO) | 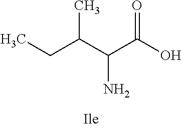 |

Since, in the method of the invention, the decarboxylation step results in a product having one C atom less than the amino acid used as starting compound, a desired diol with a given number n of C atoms will be produced from an amino acid having a number of n+1 C atoms (see table 2). Different diols may, however, be produced from the same amino acid, the amino acid being hydroxylated at different positions. As an example, 1,2-Pentanediol, 1,3-Pentanediol and 1,4-Pentanediol can all be produced from norleucine.

In preferred embodiments of the method of the invention

a) Isopentyldiol is produced from leucine, the method steps being performed in the order a, b, c, d, or

b) 1,4-Butanediol is produced from norvaline, the method steps being performed in the order a, b, c, d, or

c) 1,4-Pentanediol is produced from norleucine, the method steps being performed in the order a, b, c, d, or

d) 2-Methyl-1,4-butanediol is produced from leucine, the method steps being performed in the order a, b, c, d, or

e) 2-Methyl-1,3-propanediol is produced from valine, the method steps being performed in the order a, b, c, d, or

f) 1,3-Propanediol is produced from threonine, the method steps being performed in the order a, b, c, d, or

g) 1,3-Butanediol is produced from norvaline, the method steps being performed in the order a, b, c, d, or

h) 2-Methyl-1,3-butanediol is produced from isoleucine, the method steps being performed in the order a, b, c, d, or

i) 2-Ethyl-1,3-propanediol is produced from isoleucine, the method steps being performed in the order a, b, c, d, or

j) 1,3-Pentanediol is produced from norleucine, the method steps being performed in the order a, b, c, d.

It is preferred that genetically engineered microorganisms, preferably bacterial cells, e.g. E.-coli cells, or yeast cells, are used in the method of the invention. In order to produce a desired diol, a cell, for example a bacterial cell, is genetically engineered to express at least one heterologous enzyme necessary to catalyze steps a, b, c or d. The cell may be genetically engineered to express one, two, three or four heterologous enzymes catalyzing steps a, b. c or d. A bacterial cell, e.g. an E.-coli cell, may, for example, be engineered to express a heterologous hydroxylase and a heterologous decarboxylase. Together with an endogenous (homologous) deaminase and an endogenous (homologous) reductase (hydrogenase) a desired diol, for example 1,3-PDO, can be produced. It is preferred that the at least one heterologous enzyme that is expressed in the cell is a hydroxylase.

By using genetically engineered microorganisms as described above it is possible to produce a given diol from a common substrate like glucose. The cells can use, for example, glucose, or any other source or substrate, from which the cell can derive glucose, to synthesize an amino acid necessary for producing a desired diol, and which will then be further metabolized to the desired diol.

### BRIEF DESCRIPTION OF THE FIGURES

In the following, the invention will be described in further detail by way of example only with reference to the accompanying figures.

FIG. 1. Schematic representation of the general approach for producing diols from amino acids according to an embodiment of the method of the invention.

FIG. 2. Simplified and schematic overview of canonical and non-canonical amino acids, cellular metabolic pathways leading to the amino acids and exemplary diols (numbering see also table 2) that can be produced with the method of the invention. 3PG: 3-Phosphoglyceric acid; PEP: Phosphoenolpyruvate; PYR: pyruvate; Ace-CoA: Acetyl-CoA; KIV: 2-ketoisovalerate; KIC: 2-ketoisocaproate; OAA: Oxaloacetate; α-KG: α-ketoglutarate; CAC: Citric acid cycle.

FIG. 3 GC analysis results of recombinant strains IP01 and IP00 after 48 h of fermentation in shake flasks.

FIG. 4 Determination of IPDO concentrations produced by strains IP01 (with or without addition of leucine) and IP00.

FIG. 5. Fermentation results of recombinant strains under different conditions.

FIG. 6. GC analysis results of recombinant strains 14BDO01 and 14BDO00 after 48 h of fermentation in shake flasks.

FIG. 7. GC analysis results of recombinant strains 13BDO01 and 13BDO00 after 48 h of fermentation in shake flasks.

FIG. 8. GC analysis results of recombinant strains 14PEO01 and 14PEO00 after 48 h of fermentation in shake flasks.

FIG. 9. GC analysis results of recombinant strains MPO01 and MP000 after 48 h of fermentation in shake flasks.

FIG. 10. GC analysis results of recombinant strains MBDO01 and MBDO00 after 48 h of fermentation in shake flasks.

FIG. 11. Production of 2-methyl-1,3-butanediol, 2-ethyl-1,3-propanediol and 1,3-pentanediol in E. coli with the method of the invention. Different hydroxylases were used. 1,3-PTD=1,3-pentanediol); 2-M-1,3-BDO=2-Methyl-1,3-butanediol); 2-E-1,3-PDO=2-ethyl-1,3-propanediol.

### DETAILED DESCRIPTION OF THE EMBODIMENTS

### Examples

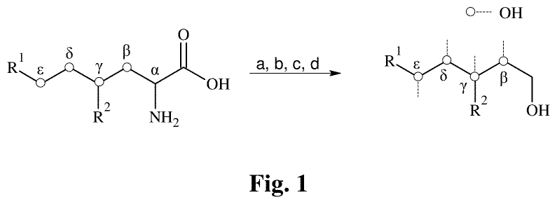

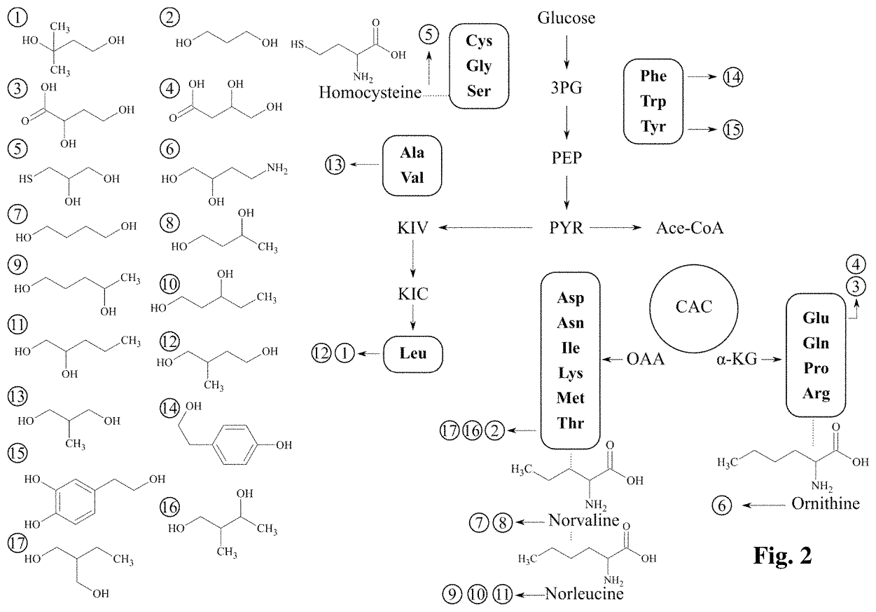

FIG. 1 schematically shows a general principle for producing diols from an alpha amino acid. In the method of the invention, the hydroxyl group (—OH) may, for example, be enzymatically attached to the beta, gamma, delta or epsilon C atom of an alpha amino acid. Possible C atoms for attaching the hydroxyl group are marked with a circle.

### Example 1. Production of Isopentyldiol (IPDO) from Leucine (Leu)

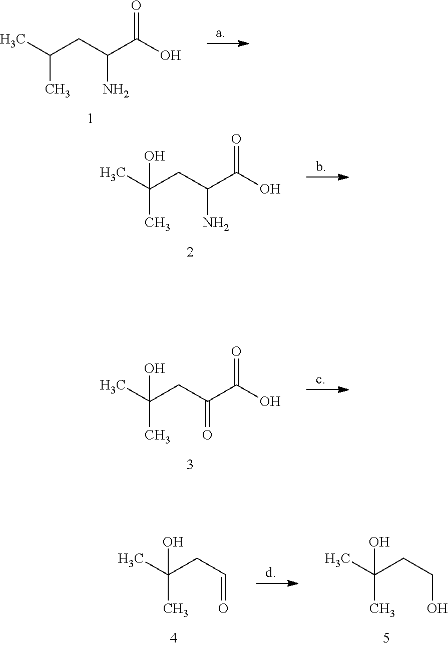

Isopentyldiol (IPDO) (5) was produced from Leucine (1) in E. coli BL21(DE3). For this purpose, leucine (Leu) 1 was hydroxylated in step a with a hydroxylase Mfl from Methylobacillus flagellates, which catalyzes the hydroxylation of leucine to 4-hydroxyleucine 2. In the following step b, 4-hydroxyleucine 2 was deaminated with amino acid deaminase (Aad) to 4-hydroxy-4-methyl-2-oxopentanoic acid 3, which, in the following step c, was decarboxylated with alpha-keto acid decarboxylase (alpha-keto acid dehydrogenase complex/pyruvate dehydrogenase complex, Kdc/Pdc) to 3-hydroxy-3-methylbutanal 4. The latter was reduced to IPDO 5 with aldehyde reductase (YqhD) in the final step d.

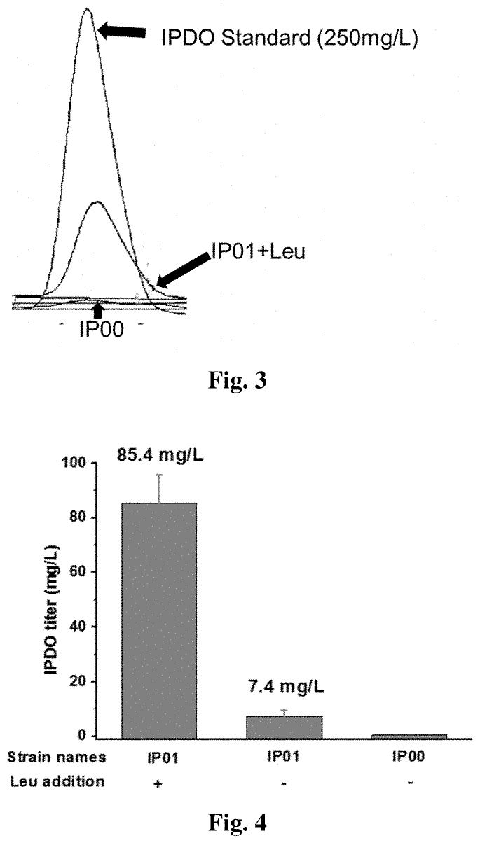

Given that Aad is a membrane-associated enzyme which is very difficult to purify in vitro, it was directly attempted to verify this pathway in vivo. We overexpressed L-isoleucine-4-hydroxylase (Mfl) from Methylobacillus flagellatus in plasmid pET28a (named as pET-mfl) The genes aad, kdc and yqhD were separately amplified from the genomes of Proteus vulgaris, Lactobacillus lactis and Escherichia coli respectively and ligated into the pZA vector to obtain pZA-aky. The two plasmids were then co-transformed into E. coli BL21(DE3), resulting in strain IP01. E. coli BL21(DE3) carrying the plasmid pZA and pET28-mfl was constructed as a control (named as IP00). Both strains were cultivated in LB medium overnight and subsequently cultivated in Fe-II medium (30 g/L Glucose, 0.5 g/L MgSO₄.7H₂O, 3 g/L KH₂PO₄, 12 g/L K₂ HPO₄, 4 g/L (NH₄)₂SO₄, 1 g/L Yeast extract, 2 g/L Monosodium citrate and 0.1 g/L FeSO₄ 7H₂O) with an inoculation rate of 1:100. 25 mM leucine was added into the medium when the OD₆₀₀ (optical density) reached approximately 0.6. In addition, IP01 strain cultivated in Fe-II medium without additional Leu was carried out in parallel. After 48 h of fermentation, GC analysis was carried out to measure the amount of IPDO in the samples. As shown in Figure. 3, a titer of 85.4 mg/L IPDO was detected from strain IP01 after 48 h of fermentation. Moreover, the IP01 strain produced 7.4 mg/L IPDO even when no external Leu was added (Figure. 4). As a control, no detectable IPDO was observed from the strain IP00. This implied the viability of IPDO production from leucine through the pathway shown above.

### Example 2. Production of 1,3-Propanediol (1,3-PDO) from Threonine (Thr)

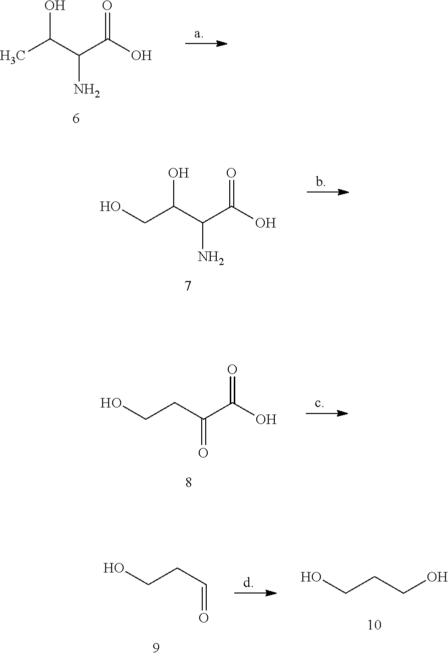

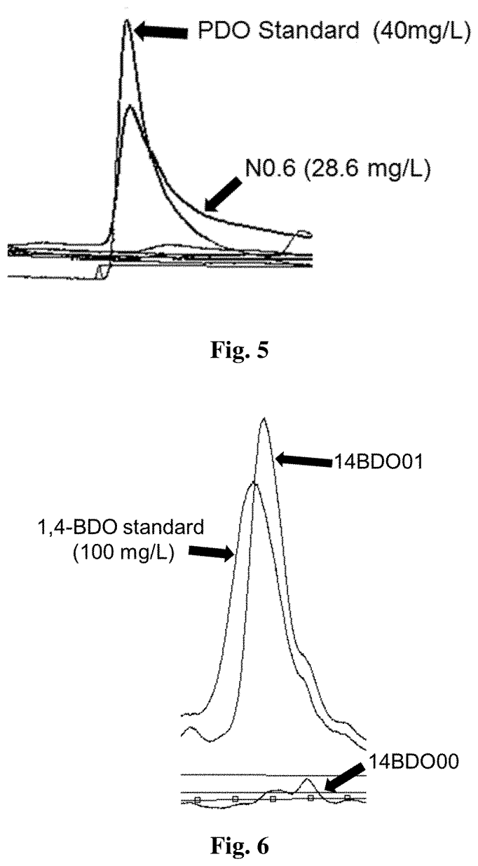

A biosynthetic route according to the invention was used for 1,3-PDO production from threonine 10 via 4-Hydroxy-threonine 7, 4-Hydroxy-α-ketobutyric acid 8 and 3-Hydroxy-propionaldehyde. Threonine hydroxylase BPE from Bordetella petrii, alcohol dehydrogenase YqhD from E. coli as well as decarboxylase Pdc or Kdc were employed. In addition, Aad is substituted by the endogenous threonine deaminase IlvA for deamination. E. coli cultures expressing these foreign genes were cultivated in Fe-II medium and 25 mM threonine were added into the medium after 2 h of induction by IPTG. When the four enzymes were overexpressed simultaneously (PD1 and PD2 strain) no 1,3-PDO was detected. We assumed this was attributed to the low K_{cat} of BPE which resulted in the majority of threonine was directly converted into α-ketobutyric acid without hydroxylation. Thus, we overexpressed three pathway enzymes in the recombinant strains and the deamination of 4-hydroxyo-threonine was fulfilled by endogenous IlvA. Results showed strain PD4 harboring kdc (No. 6 in Table. 3) produced 28.6 mg/L 1,3-PDO while no 1,3-PDO was generated in strain PD3 harboring pdc (Figure. 5). This indicated that compared to pdc, kdc presented higher catalytic activity towards the intermediate 4-hydroxy-2-ketobutyrate. To test whether 1,3-PDO could be synthesized in the order b,a,c,d (Deamination-Hydroxylation-Decarboxylation-Reduction), we cultivated PD3 and PD4 strains and added 25 mM α-ketobutyric acid instead of threonine to the medium (No. 3 and NO. 5 in Table. 3). It was found 1,3-PDO couldn't be generated from α-ketobutyric acid. This is probably because that BPE presented little or none activity for hydroxylation of α-ketobutyric acid.

TABLE 3
Information of genes employed to overexpress in the
recombinant strains and the compounds added into the
medium (Thr: threonine: α-kb: α-ketobutyric acid)

| No. | 1 | 2 | 3 | 4 | 5 | 6 |
|---|---|---|---|---|---|---|
| Strains | PD1 | PD2 | PD3 | PD3 | PD4 | PD4 |
| hydroxylase | BPE | BPE | BPE | BPE | BPE | BPE |
| deaminase | IlvA | IlvA | — | — | — | — |
| decarboxylase | PDC | KDC | PDC | PDC | KDC | KDC |
| additives | Thr | Thr | α-kb | Thr | α-kb | Thr |

### Example 3. Production of 1,4-Butanediol (1,4-BDO) from Norvaline (Nva)

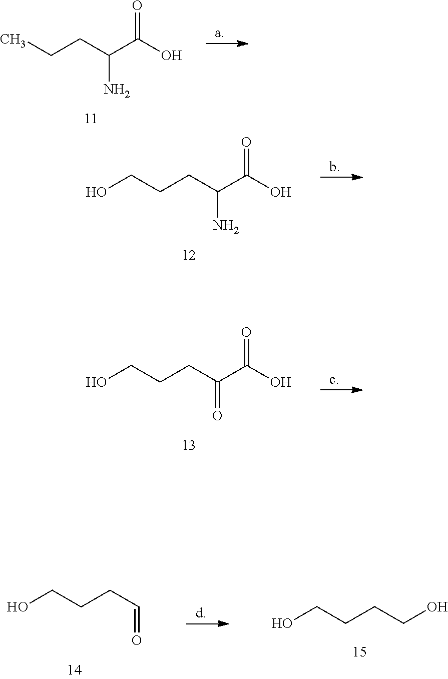

For the production of 1,4-Butanediol (1,4-BDO) from Norvaline (Nva), E. coli BL21(DE3) was genetically engineered to express a heterologous leucine hydroxylase GriE from Mycobacterium tuberculosis and decarboxylase Kdc from Lactobacillus lactis. In a first step (a.), norvaline 11 was hydroxylated by the heterologous hydroxylase GriE to form 5-Hydroxy-norvaline 12. In a following step (b.) the resulting 5-Hydroxy-norvaline was deaminated with amino acid deaminase (Aad) to 5-Hydroxy-2-oxopentanoic acid 13. Decarboxylation of the latter in the following step (c.) resulted in 4-Hydroxy-butyraldehyde 14, which was reduced in step d. by the endogenous reductase (YqhD) to the desired diol 1,4-BDO 15.

We overexpressed leucine hydroxylase from Mycobacterium tuberculosis (GriE) in plasmid pET28a (named as pET-griE) The genes aad, kdc and yqhD were separately amplified from the genome of Proteus vulgaris, lactobacillus lactis and Escherichia coli respectively and ligated into the pZA vector to obtain pZA-aky. The two plasmids were then co-transformed into E. coli BL21(DE3), resulting in 14BDO01. E. coli BL21(DE3) carrying the plasmid pZA and pET28-griE was constructed as a control (named as 14BDO00) and both strains were cultivated in LB medium overnight and subsequently cultivated in Fe-II medium with the inoculation rate of 1:100. 25 mM norvaline was added into the medium when the OD₆₀₀ (optical density) reached approximately 0.6. After 48 h of fermentation, samples were enriched for 10 times to measure the amount of 1,4-BDO by GC analysis. As shown in Figure. 6, a titer of 10.8 mg/L 1,4-BDO was detected from strain 14BDO01 after 48 h of fermentation. As a control, no detectable 1,4-BDO was observed from the strain 14BDO00. This implied the viability of 1,4-BDO production from norvaline through the general pathway.

### Example 4. Production of 1,3-Butanediol (1,3-BDO) from Norvaline (Nva)

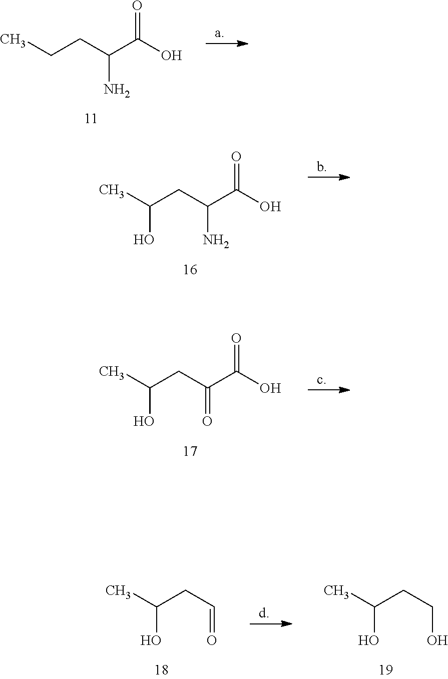

For the production of 1,3-Butanediol (1,3-BDO) from Norvaline (Nva), E. coli BL21(DE3) was genetically engineered to express Mfl from Methylobacillus flagellatus and decarboxylase Kdc from Lactobacillus lactis. In a first step (a.), norvaline 11 was hydroxylated by Mfl to form 4-Hydroxy-norvaline 16. In a following step (b.) the resulting 4-Hydroxy-norvaline was deaminated with amino acid deaminase (Aad) to 4-Hydroxy-2-oxopentanoic acid 17. Decarboxylation of the latter in the following step (c.) resulted in 3-Hydroxy-butyraldehyde 18, which was reduced in step d. by the endogenous reductase (YqhD) to the desired diol 1,3-BDO 19.

We overexpressed Mfl in plasmid pET28a (named as pET-mfl) The gene aad, kdc and yqhD were separately amplified from the genome of Proteus vulgaris, Lactobacillus lactis and Escherichia coli and ligated into pZA vector to obtain pZA-aky. The two plasmids were then co-transformed into E. coli BL21(DE3), resulting 13BDO01. E. coli BL21(DE3) carrying the plasmid pZA and pET28-mfl was constructed as a control (named as 13BDO00) and both strains were cultivated in LB medium overnight and subsequently cultivated in Fe-II medium with the inoculation rate of 1:100. 25 mM norvaline was added into the medium when the OD₆₀₀ (optical density) reached approximately 0.6. After 48 h of fermentation, samples were enriched for 10 times to measure the amount of 1,3-BDO by GC analysis. As shown in FIG. 7, a titer of 8.2 mg/L 1,3-BDO was detected from the strain 13BDO01. As a control, no detectable 1,3-BDO was observed from the strain 13BDO00. This confirms the feasibility of 1,3-BDO production from norvaline through the general pathway.

### Example 5. Production of 1,4-Pentanediol (1,4-PTD) from Norleucine (Nle)

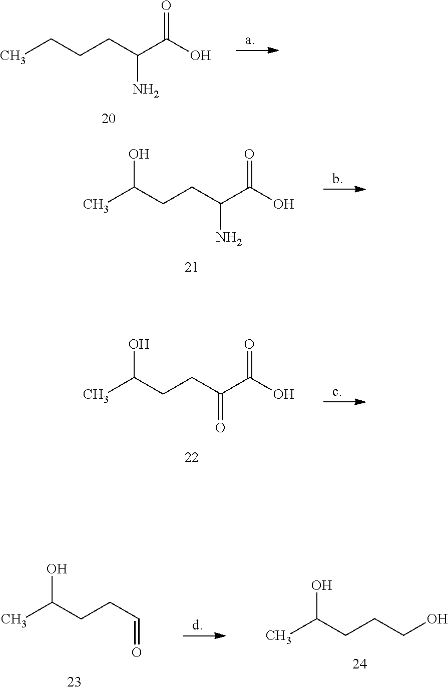

For the production of 1,4-Pentanediol from norleucine. E. coli BL21(DE3) was genetically engineered to express a heterologous leucine hydroxylase GriE from Mycobacterium tuberculosis and decarboxylase Kdc from Lactobacillus lactis. In a first step (a.), norleucine 20 was hydroxylated by the heterologous hydroxylase GriE to form 5-Hydro-Norleucine 21. In a following step (b.) the resulting 5-Hydroxy-norleucine was deaminated with amino acid deaminase (Aad) to 5-Hydroxy-2-oxyhexanoic acid 22. Decarboxylation of the latter in the following step (c.) resulted in 4-Hydroxy-valeraldehyde 23, which was reduced in step d. by the endogenous reductase (YqhD) to the desired diol 1,4-Pentanediol 24.

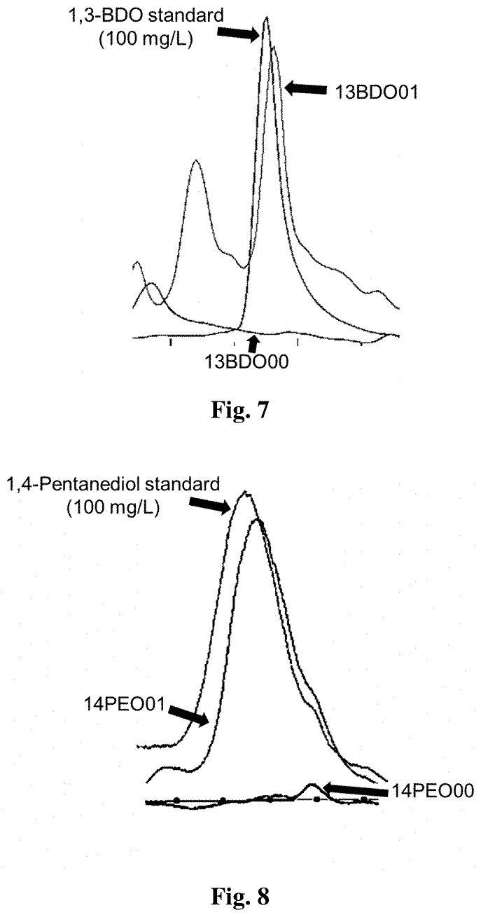

We overexpressed leucine hydroxylase from Mycobacterium tuberculosis (GriE) in plasmid pET28a (named as pET-griE) The gene aad, kdc and yqhD were separately amplified from the genome of Proteus vulgaris, lactobacillus lactis and Escherichia coli and ligated into pZA vector to obtain pZA-aky. The two plasmids were then co-transformed into E. coli BL21(DE3), resulting 14PEO01. E. coli BL21(DE3) carrying the plasmid pZA and pET28-griE was constructed as a control (named as 14PEO00) and both strains were cultivated in LB medium overnight and subsequently cultivated in Fe-II medium with the inoculation rate of 1:100. 25 mM norleucine was added into the medium when the OD₆₀₀ (optical density) reached approximately 0.6. After 48 h of fermentation, samples were enriched for 10 times to measure the amount of 1,4-pentanediol by GC analysis. As shown in Figure. 8, a titer of 8.9 mg/L 1,4-pentanediol was detected from strain 14PEO01 after 48 h of fermentation. As a control, no detectable 1,4-pentanediol was observed from the strain 14PEO00. This implied the viability of 1,4-pentanediol production from norleucine through the general pathway.

### Example 6. Production of 2-Methyl-1,3-Propanediol (2-M-1,3-PDO, or MPO) from Valine

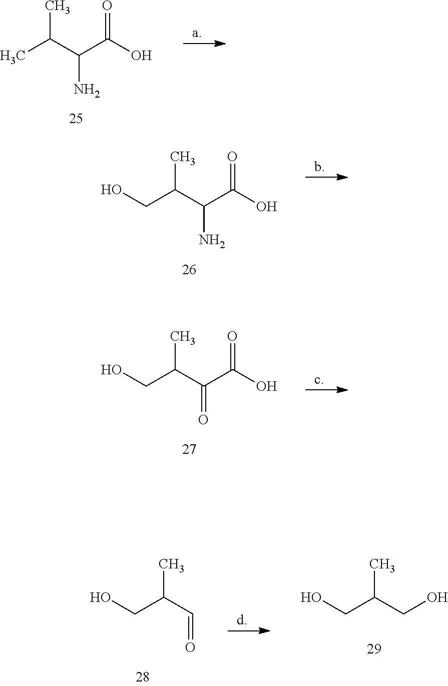

For the production of 2-Methyl-1,3-propanediol (MPO) from valine, E. coli BL21(DE3) was genetically engineered to express Mfl from Methylobacillus flagellatus and decarboxylase Kdc from Lactobacillus lactis. In a first step (a.), valine 25 was hydroxylated by Mfl to form 4-Hydroxy-valine 26. In a following step (b.) the resulting 4-Hydroxy-valine was deaminated with amino acid deaminase (Aad) to 3-Methyl-4-Hydroxy-2-oxybutyric acid 27. Decarboxylation of the latter in the following step (c.) resulted in 2-Methyl-3-Hydroxy-propionaldehyde 28, which was reduced in step d. by the endogenous reductase (YqhD) to the desired diol MPO 29.

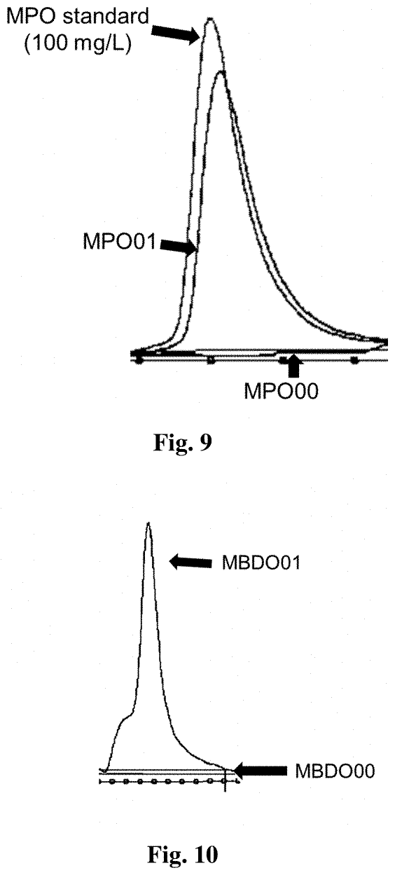

We overexpressed Mfl in plasmid pET28a (named as pET-mfl). The gene aad, kdc and yqhD were separately amplified from the genome of Proteus vulgaris, Lactobacillus lactis and Escherichia coli and ligated into pZA vector to obtain pZA-aky. The two plasmids were then co-transformed into E. coli BL21(DE3), resulting MPO01. E. coli BL21(DE3) carrying the plasmid pZA and pET28-mfl was constructed as a control (named as MP000) and both strains were cultivated in LB medium overnight and subsequently cultivated in Fe-II medium with the inoculation rate of 1:100. 25 mM valine was added into the medium when the OD₆₀₀ (optical density) reached approximately 0.6. After 48 h of fermentation, samples were collected to measure the amount of MPO by GC analysis. As shown in Figure. 9, a titer of 105.5 mg/L MPO was detected from strain MPO01 after 48 h of fermentation. As a control, no detectable MPO was observed from the strain MP000. This implied the viability of MPO production from valine through the general pathway.

### Example 7. Production of 2-Methyl-1,4-Butanediol (2-M-1,4-BDO) from Leucine

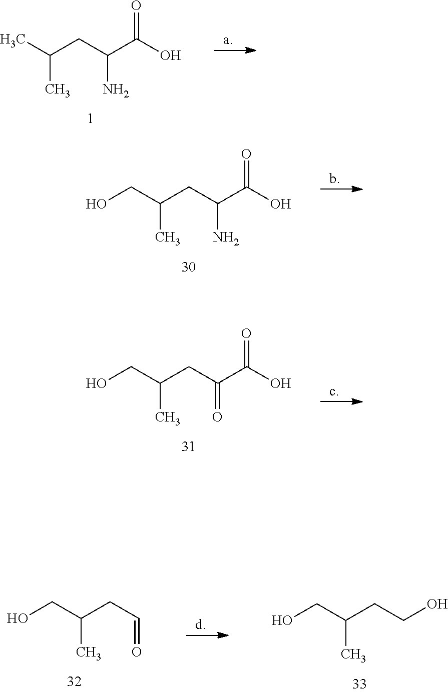

For the production of 2-Methyl-1,4-Butanediol from leucine, E. coli BL21(DE3) was genetically engineered to express a heterologous leucine hydroxylase GriE from Mycobacterium tuberculosis and decarboxylase Kdc from Lactobacillus lactis. In a first step (a.), leucine 1 was hydroxylated by GriE to form 5-Hydroxy-leucine 30. In a following step (b.) the resulting 5-Hydroxy-leucine was deaminated with amino acid deaminase (Aad) to 4-Methyl-5-Hydroxy-2-oxopentanoic acid 31. Decarboxylation of the latter in the following step (c.) resulted in 3-Methyl-4-Hydroxy-butyraldehyde 32, which was reduced in step d. by the endogenous reductase (YqhD) to the desired diol 2-Methyl-1,4-Butanediol 33.

We overexpressed GriE in plasmid pET28a (named as pET-griE) The gene aad, kdc and yqhD were separately amplified from the genome of Proteus vulgaris, Lactobacillus lactis and Escherichia coli and ligated into pZA vector to obtain pZA-aky. The two plasmids were then co-transformed into E. coli BL21(DE3), resulting MBDO01. E. coli BL21(DE3) carrying the plasmid pZA and pET28-griE was constructed as a control (named as MBDO00) and both strains were cultivated in LB medium overnight and subsequently cultivated in Fe-II medium with the inoculation rate of 1:100. 25 mM leucine was added into the medium when the OD₆₀₀ (optical density) reached approximately 0.6. After 48 h of fermentation, samples were collected to measure the amount of 2-Methyl-1,4-Butanediol by GC analysis. As can be seen in FIG. 10, a large peak was found in the sample MBDO01 and the retention time of this peak was 9.26 min. As a control, no detectable MPO was observed from the strain MBDO00. Although the commercial standard of 2-Methyl-1,4-BDO is not available, it was quite possible the peak represents 2-Methyl-1,4-Butanediol. However, the absolute quantity of this product is uncertain.

### Example 8 Production of 2-methyl-1,3-butanediol (2-M-1,3-BDO), 2-ethyl-1,3-propanediol (2-Et-1,3-PDO) and 1,3-pentanediol (1,3-PTD) in E. coli

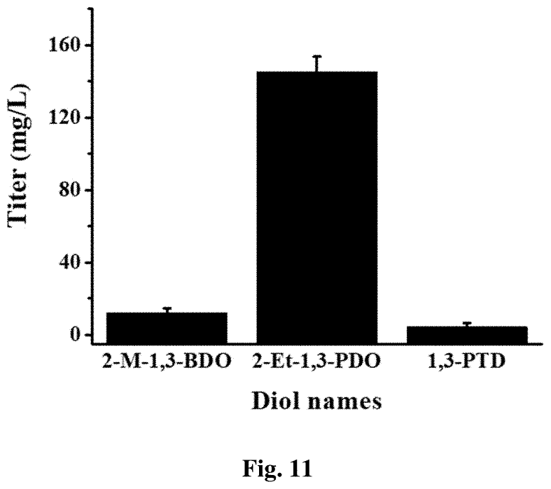

In order to confirm the versatility of the method of the invention, hydroxylases from various species were employed to produce diverse diols from different substrate amino acids in E. coli. Specifically, hydroxylases HilB from Pantoea ananatis AJ13355, HilA from Pantoea ananatis AJ13355 and Mfl from Methylobacillus flagellatus KT were separately ligated in pET28a plasmid, generating pET-hilB, pET-hilA and pET-mfl. L-amino acid deaminase, α-keto acid decarboxylase and aldehyde reductase were co-expressed in the plasmid pZA. Two plasmids carrying pathway enzymes were then co-transformed into E. coli BL21(DE3), resulting in strains 2M13BDO01, 2E13PDO01 and 13PTD01, respectively. Strains 2M13BDO01, 2E13PDO01 and 13PTD01 were cultivated in LB medium overnight and subsequently cultivated in modified FM-II medium (30 g/L glucose, 0.5 g/L MgSO₄.7H₂O, 3 g/L KH₂PO₄, 12 g/L K₂HPO₄, 4 g/L (NH₄)₂SO₄, 1 g/L yeast extract, 2 g/L monosodium citrate, 1 mg/L thiamine, 100 μg/L biotin, 1 mL/L US^{Fe} trace element solution (Buhler, B., Bollhalder, I., Hauer, B., Witholt, B., Schmid, A. 2003, Use of the two-liquid phase concept to exploit kinetically controlled multistep biocatalysis, Biotechnology and bioengineering 81, 683-694), and 0.2 g/L FeSO₄.7H₂O) with an inoculation rate of 1:100. 25 mM substrate amino acid was added into the medium when the OD₆₀₀ (optical density) reached approximately 0.6. Results indicated that a titer of 12.1 mg/L 2-Me-1,3-BDO (2-methyl-1,3-butanediol) were produced by 2M13BDO01 in modified FM-II medium supplemented with 25 mM isoleucine, 145.2 mg/L 2-Et-1,3-PDO (2-ethyl-1,3-propanediol) were produced by 2E13PDO01 in modified FM-II medium supplemented with 25 mM isoleucine and 4.2 mg/L 1,3-PTD (1,3-pentanediol) were produced by 13PTD01 in modified FM-II medium supplemented with 25 mM norleucine. (FIG. 11).

1-8. (canceled)

9. A method for the biologic synthesis of a diol from an amino acid, the method comprising: the steps of
a. enzymatically hydroxylating the amino acid,
b. enzymatically deaminating the amino acid, the deamination resulting in the formation of an oxo group at the position of the amino group,
c. enzymatically decarboxylating the amino acid, and
d. enzymatically hydrogenating the oxo group resulting from the deamination reaction.

10. The method according to claim 9, wherein the amino acid is an alpha amino acid.

11. The method according to claim 9, wherein the amino acid is enzymatically hydroxylated at a C atom of the side chain.

12. The method according to claim 9, wherein the method steps are performed in the order a, b, c, d.

13. The method according to claim 9, wherein the amino acid is an aliphatic amino acid.

14. The method according to claim 13, wherein
a) Isopentyldiol is produced from leucine, the method steps being performed in the order a, b, c, d, or
b) 1,4-Butanediol is produced from norvaline, the method steps being performed in the order a, b, c, d, or
c) 1,4-Pentanediol is produced from norleucine, the method steps being performed in the order a, b, c, d, or
d) 2-Methyl-1,4-butanediol is produced from leucine, the method steps being performed in the order a, b, c, d, or
e) 2-Methyl-1,3-propanediol is produced from valine, the method steps being performed in the order a, b, c, d, or
f) 1,3-Propanediol is produced from threonine, the method steps being performed in the order a, b, c, d, or
g) 1,3-Butanediol is produced from norvaline, the method steps being performed in the order a, b, c, d, or
h) 2-Methyl-1,3-butanediol is produced from isoleucine, the method steps being performed in the order a, b, c, d, or
i) 2-Ethyl-1,3-propanediol is produced from isoleucine, the method steps being performed in the order a, b, c, d, or
j) 1,3-Pentanediol is produced from norleucine, the method steps being performed in the order a, b, c, d.

15. The method of claim 9, wherein the diol is synthesized by a cell, preferably a bacterial cell, genetically engineered to express at least one heterologous enzyme catalyzing one of the steps a, b, c or d.

16. The method of claim 15, wherein the genetically engineered cell is an E.-coli cell.
